(19) United States
(12) Patent Application Publication
(10) Pub. No.: US 2015/0216839 A1
(43) Pub. Date: Aug. 6, 2015

(54) COMPOSITIONS OF BIOACTIVE POLYELECTROLYTES FROM HUMIFIED ORGANIC MATERIALS AND USES THEREOF

(71) Applicant: BPW SCIENCES, LP, King Of Prussia, PA (US)

(72) Inventor: Gary W. Black, SR., Pottstown, PA (US)

(73) Assignee: BPW Sciences, LP, King of Prussia, PA (US)

(21) Appl. No.: 14/429,845

(22) PCT Filed: Sep. 19, 2013
(86) PCT No.: PCT/US13/60627

§ 371 Date: Mar. 20, 2015

Related U.S. Application Data
(60) Provisional application No. 61/703,499, filed on Sep. 20, 2012.

(51) Int. Cl. A61K 31/352; A61Q 19/08; A61K 31/4174; A61K 31/4412; A61K 8/49; A61K 31/137

(52) U.S. Cl. CPC A61K 31/352; A61K 8/498; A61K 31/137; A61K 31/4174; A61K 31/4412; A61Q 19/08; A61K 2800/412

(57) ABSTRACT

Topical compositions containing an isolated BP fraction and an active agent, and uses of the composition to improve a skin condition are described.

### BACKGROUND OF THE INVENTION

Current dermatologists and cosmetologists have directed their efforts toward improving the appearance of the undesirable effects of wrinkles, lines, and discoloration of the skin by various methods, including providing growth stimulants, such as irritants or chemical peels, to the epidermal cells. While these procedures have provided some variations of success, they are met with the dangers of scarring, improper healing, and poor cell replenishment. More importantly, these avenues do not address the underlying major cause of poor skin, the deterioration of the supporting dermal layer. Other modes of treatment include using topical treatments to carry key ingredients and nutrition to the dermal layers of skin or using collagen or fat injections to smooth wrinkled skin. These choices require frequent use, can be costly, and only provide temporary corrections.

Certain humic substances (HS), such as peat-derived bioactive products, have been used for treating skin and other conditions. HS are among the most widely distributed natural products on the surface of the earth, and are the major organic components of soil (humus), lakes, rivers and geological deposits such as peat, leonardite, lignite (brown coal) and organic clays. However, the efficacy of HS related compositions varies depending on the source and quality of the humified organic materials (HMOs) used. HOMs vary in composition based on their sources, e.g., location, deposit type, depth, age, etc. They can include a vast and complex array of polymers that range in molecular weights from 60 to 300,000 Da and lengths from a few nanometers to several microns, with different biological activities. In additions, potential contaminations in the HOM preparations, such as heavy metals (e.g., chromium, mercury, lead), metals (e.g., iron, aluminum, silica), as well as other toxic or undesirable organic or inorganic matters, also cause concerns over broad applications of HS related compositions in skin treatment.

The contaminants or undesirable ingredients include, but are not limited to, heavy metals such as chromium, mercury and lead, and metals such as iron, aluminum and silica, as well as other toxic or undesirable organic or inorganic matters.

Therefore, a need exists for a method and composition that provide efficacious and more lasting relief from unwanted skin conditions including, but not limited to, wrinkles, nail fungus, and ringworm.

### BRIEF SUMMARY OF THE INVENTION

It is now discovered that an isolated bioactive polyelectrolyte (“BP”) fraction, such as an isolated fulvic acid and/or humic acid fraction, obtained from naturally occurring HOM possesses a wide range of beneficial characteristics in improving a variety of skin conditions. The isolated BP fraction can be used to facilitate the treatment of skin cell deterioration, reduction or elimination of aging appearance of skin, treatment of fungal infections, and many other skin treatments.

In one general aspect, the present invention relates to a topical composition for improving a skin condition. The composition comprises a pharmaceutically or cosmetically acceptable carrier, at least one active agent, and an effective amount of an isolated bioactive polyelectrolyte (BP) fraction, comprising an isolated fulvic acid fraction, an isolated humic acid fraction, or a mixture of isolated fulvic acid and humic acid fraction, wherein the isolated fulvic acid fraction consists essentially of ionic nanocrystalline fulvic acid molecules, and the isolated humic acid fraction consists essentially of colloidal humic acid particles having an average particle size of about 1 to 10 microns in diameter.

Another general aspect of the present invention relates to a method for improving a skin condition in a subject. The method comprises topically applying to a skin area of the subject in need of the improvement a topical composition according to an embodiment of the present invention.

Other aspects, features and advantages of the invention will be apparent from the following disclosure, including the detailed description of the invention and its preferred embodiments and the appended claims.

### DETAILED DESCRIPTION OF THE INVENTION

Various publications, articles, and patents are cited or described in the background and throughout the specification; each of these references is herein incorporated by reference in its entirety. Discussion of documents, acts, materials, devices, articles, or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications, and publications cited herein are incorporated by reference as if set forth fully herein. It must be noted that as used herein and in the appended claimed, the singular forms “a,” “an,” and “the” include plural references unless the context clearly dictates otherwise.

Humified Organic Matter (HOM) is found throughout the world, commonly in humilite, leonardite, peat, brown coal, and lignite. These HOM deposits are generally found adjacent to peat bogs and certain coal deposits, such as sub-bituminous coal, and contain varying purity and desired characteristics. HOM deposits have some general or typical characteristics, including the presence of varying amounts of humic substances (HS). Each of the deposits are specifically unique in their overall make up of cation exchange capacity (“CEC”) potential, polyelectrolyte composition, minerals content, amino acid fractions, and polymer and molecular specifics. HOM contains a complex mixture of organic molecules, such as bioactive polyelectrolytes (BPs).

As used herein, the term “bioactive polyelectrolyte” or “BP” refers to any bioactive polymer whose repeating units bear an electrolyte group, as well as the salts and esters of the bioactive polymer. BPs can have a wide and valuable range of beneficial uses in humans, other animals and plants. BPs can be made up of five (5) basic elements: carbon, hydrogen, nitrogen, oxygen and sulfur, while carbon and oxygen being the main components. The principal organic groups of BPs include, for example, phenolic, carboxylic, OH, aliphatic CH, carbonyl, conjugated carboxyl, aromatic CH₂ or CH₃, ionic carboxyl and possibly others. BPs can be classified and, to some extent, identified by their degree of polymerization, molecular weight and atomic particle size, characteristics that appear to be dictated by the extent and type of humification processes that produced the BPs.

Examples of BP include, but are not limited to, humic acid (HA), fulvic acid (FA), humin and ulmic acid (UA). In general, fulvic acid comprises low-molecular weight polymeric compounds, while humic acid comprises high molecular weight polymeric compounds. The humic and fulvic acid fractions of BP are a combination of colloids and nano-crystalline materials. While their exact structures are still not yet fully characterized, the HA and FA have been shown to have excellent bioactive capabilities for living matters.

The molecular sizes of BPs can be, for example, 50,000 Daltons (“Da”) to 160,000 Da for UA, 3,000-50,000 Da for HA, and 150-13,000 Da for FA.

As used herein, the term “humic acid” or “HA” refers to a fraction of humic substances that is not soluble in water at low pH, e.g., below about 2, but soluble at higher pH, e.g., about 6 or higher. It is insoluble in an acid solution, methyl ethyl ketone (MEK) and methyl alcohol (MA), but is soluble in an alkali solution. HA comprises a mixture or collection of different acids containing carboxyl and phenolate groups, some of which are based on a motif of aromatic nuclei with phenolic and carboxylic substitutions that are linked together. HA can have one or more of quinone, phenol, catechol and sugar moieties.

The proposed structures of HA contain free and bound phenolic OH groups, quinine structures, nitrogen and oxygen as bridge units and COOH groups placed variously on aromatic rings (Stevenson F. J., 1982, Humua Chemistry: Genesis, Composition, Reactions, John Wiley & Sons, Interscience 443p. New York 1982). Some of the previously proposed HA structures are illustrated in Formula (I) and Formula (II) below.

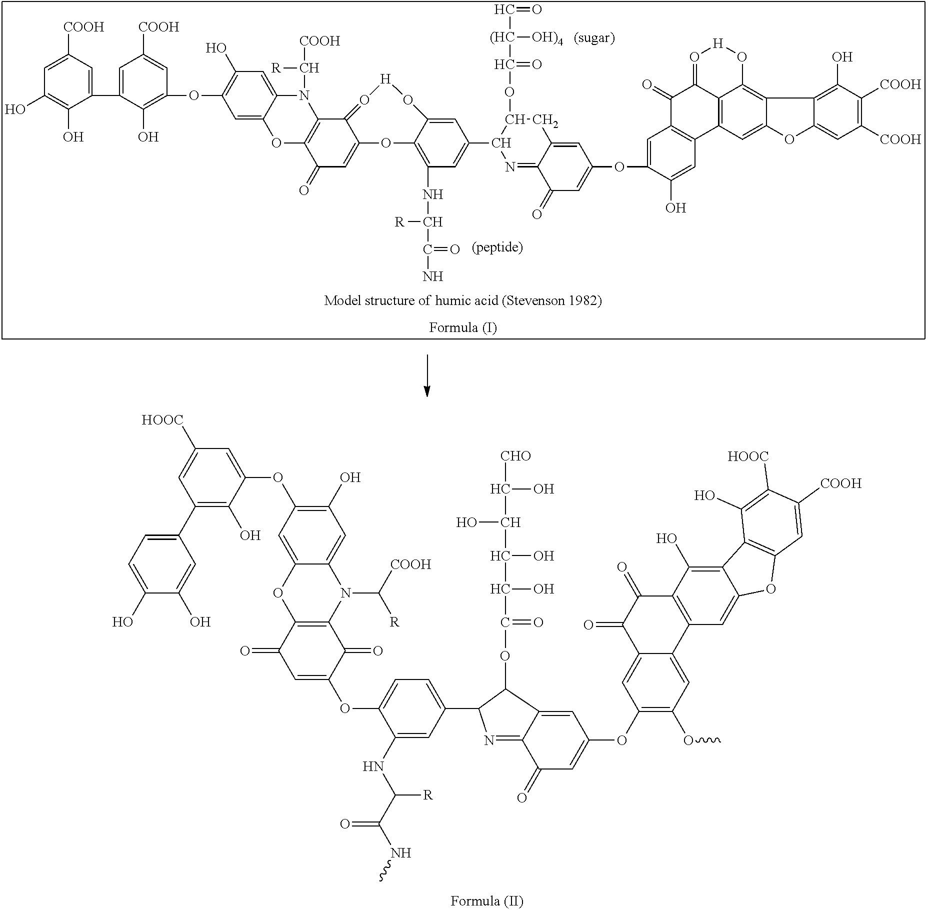

(F. J. Stevenson (1994) Humus Chemetry: Composition, Genesis, Reactions, John Wiley & Sons, New York, also as published in Wikipidia Free Encyclopedia, 2009).

HA behaves functionally as a dibasic acid or tribasic acid. It can form complexes with ions such as Mg²⁺, Ca²⁺, Fe²⁺ and Fe³⁺. As used herein, the term “HA” encompasses the esters, salts or ion complexes of humic acid.

As used herein, the term “fulvic acid” or “FA” refers to a fraction of humic substances that is soluble in water under all pH conditions. It is also soluble in MEK, MA, and acids. It generally has a yellow (fulvus) to yellow-brown color. FA comprises a mixture or collection of different acids containing carboxyl and phenolate groups.

The proposed structure of FA contains both aromatic and aliphatic structures that are extensively substituted with oxygen-containing functional groups. A previously proposed FA structure is illustrated in Formula (III), see Buffle J., Greter F. L., Haerdi W., 1977, Measurements of Complexation Properties of Humic and Fulvic Acids in Natural Water, With Lead & Copper Ion-Selective Electrodes. Anal. Chem. 49: 216-222:

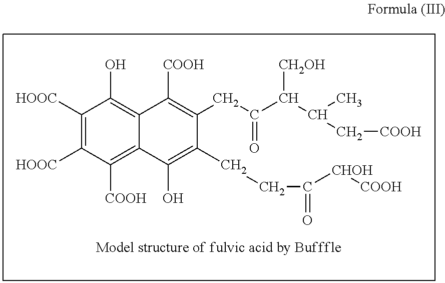

As used herein, the term “FA” encompasses the esters, salts or ion complexes of fulvic acid.

As used herein, the term “ulmic acid” or “UA” refers to a fraction of humic substances that is soluble in alkali and methyl ketone, but is insoluble in methyl alcohol. The term “UA” encompasses a mixture or collection of high molecular weight polymers, including the esters, salts or ion complexes of ulmic acid.

As used herein, the term “humin” refers to a fraction of humic substances that is insoluble in water at all pH. The term “humin” encompasses a mixture or collection of high molecular weight polymers, including the ester or salt forms of the polymers.

As used herein, an “isolated BP fraction” is substantially free of the non-BP substances present in the source where the BP fraction is isolated. An isolated BP fraction can be an isolated fraction of humic substances, such as an isolated FA fraction that is substantially free of the non-FA substances present in the source where the FA is isolated, an isolated HA fraction that is substantially free of the non-HA substances present in the source where the HA is isolated, etc. An isolated BP fraction can also contain isolated two or more fractions of humic substances, such as two or more of UA, HA, FA and humin fractions, that is substantially free of the other substances. A BP fraction is “substantially free of” the non-BP substances when there is less than about 30%, 20%, 10%, or 5%, and preferably less than 1%, by dry weight, of the non-BP substances (also referred to herein as “contaminating substances”).

An improved method for isolating BPs from HOMs have been described in U.S. Provisional Patent Application No. 61/521,172, the content of which is herein incorporated by reference in its entirety. The improved method for obtaining an isolated BP fraction from an HOM comprises:

providing an aqueous slurry comprising the HOM;

applying an electromagnetic field to the aqueous slurry to obtain an electromagnetic field treated HOM; and

isolating the BP fraction from the electromagnetic field treated HOM.

Preferably, the electromagnetic field applied to the aqueous slurry comprises a pulsed electromagnetic signal.

By adjusting or tuning the electromagnetic signal to which the HOM slurry is exposed to, separation and isolation of desirable BPs within the HOM is made possible. BP factions that have not been produced by the conventional methods can be isolated or prepared reproducibly by methods according to embodiments of the present invention.

For example, when the HA and FA fractions are separated by a conventional acid/alkaline method, there is some cross contamination or pollution of the fractions. The FA fraction still appears to be colloidal in nature because enough of the humic molecules remain in the principally fulvic fraction. However, when extracted by the improved method, the increased purity of each of the HA and FA fractions allows more accurate display of the nature of the fractions, i.e., HA is colloidal in nature and FA is ionic nano-crystalline in nature. Humic acid fractions extracted by the improved method are true colloidal, they do not dissolve in water, remaining as electrically suspended molecules, or clusters of molecules. The HA fraction containing HA particles having an average particle size of about 1 to 10 microns. Fulvic acid fractions extracted by the improved method are true solutions, i.e., a highly electrically charged ionic, nano-crystalline solution containing FA molecules about 1 to several angstroms in size.

Using the improved method, BPs, not restricted to a specific type, were extracted from HOM deposits found in Alberta, Canada. They possess larger cation exchange capacity (CEC) potentials, i.e., about 200 to 800 milliequevalents per 100 grams at a pH of 7, than those extracted from other HOM deposits, such as peat, brown coal and leonardite that typically have shown CEC potentials of from 50 to 150 milliequevalents per 100 grams at a pH of 7.

It has been discovered in the present invention that an isolated BP fraction containing one or more of BPs, such as HAs, FAs, UAs and humans, prepared by the improved method involving an electromagnetic field, provides effective improvement or treatment of a skin condition. The skin condition can be any cosmetic, dermatological, or other conditions, anomalies and disorders of cutaneous system and other tissues and systems, including, but not limited to, onychomycosis (nail fungus), ringworm, other fungal infections; skin damage, wrinkling, old age skin condition, discoloration, and age spots; psoriasis; eczema; shingles; rosacea; dryness; irritated skin from sun burn, sun spots and other damage from sun exposure; various rashes, insect bites, and other skin conditions resulting from environmental exposures.

In one general aspect, the present invention relates to a topical composition for improving a skin condition. The composition comprises a pharmaceutically or cosmetically acceptable carrier, at least one active agent, and an effective amount of an isolated bioactive polyelectrolyte (BP) fraction, comprising an isolated fulvic acid fraction, an isolated humic acid fraction, or a mixture thereof, wherein the isolated fulvic acid fraction consists essentially of ionic nanocrystalline fulvic acid molecules, and the isolated humic acid fraction consists essentially of colloidal humic acid particles having an average particle size of about 1 to 10 microns in diameter

While not wishing to be bound by theories, it is believed that the isolated BPs, with increased purity, low ash content, and/or higher CEC are superior because they can be used as transporters, carriers, binders, cation exchange media, nutritional sources, electrolytes, and molecular sieves, which are associated with promoting skin health and care. While the mode of action of the present composition is not completely or wholly understood, it is believed that using compositions according to embodiments of the present invention, one or more active agents or ingredients are bound and transported biochemically to the cells via an isolated BP more readily and more completely than would be available if not using the BP. Additionally, the unique molecular structure of the isolated BP may have caused the CEC potential and molecular size of one or more ingredients in the composition, including one or more active agents, to be modified. This thereby reduces the crystalline structure of these materials to a nano-sized particle with a highly electrolytic potential, causing them to be readily transported through the skin layers and into the cell and blood vessels, thereby carrying the active nutrients directly to the bio-energy source for use, and for conversion via chelation and enzymatic enhancement that results in cell nitrification. A composition according to an embodiment of the present invention is believed to cooperate to stimulate fibroblast proliferation and to promote their production of collagen and elastin, further promoting the supporting role of the associated dermal tissue. It is also believed that compositions according to embodiment of the present invention provides a sterically stabilized reversible cross-linked polyelectrolyte complex with one or more active agents to thereby facilitate the application of the active agents.

The present invention also relates to the treatment or improvement of a skin condition in a subject, comprising administering to a skin area in need of the treatment a topical composition according to an embodiment of the present invention. The topical composition can be administered as a lotion, cream, balm, spray, solution, or scrub.

One or more isolated BPs, such as HA, FA, and UA, can be used as electrochemical reactants, carriers, chelators, bio-stimulants, mineral sources, binder transporters, and/or electrolytes into which active and non-active agents, such as minerals, vitamins, nutrients, enzymes, and organic and inorganic chemicals, can be added to produce a bio-available nutritional and/or healing formulation that can be supplied in a myriad of ways such as solutions, lotions, creams, and sprays to revive, rejuvenate and aid in improving a skin condition, such as in healing a skin disease, reducing skin wrinkles, lines, and discolorations, etc.

As used herein, the term “subject” means any animal, preferably a mammal, most preferably a human, to whom will be or has been administered compounds or topical formulations according to embodiments of the invention. The term “mammal” as used herein, encompasses any mammal. Preferably, a subject is in need of, or has been the object of observation or experiment of, treatment or prevention of a skin condition.

As used herein, an “effective amount” means the amount of an isolated BP fraction that elicits the desired biological or medicinal response in a tissue system, an animal or human that is being sought by a researcher, veterinarian, medical doctor or other clinician, which includes improving the treatment or alleviation of the skin condition being treated.

The carriers useful for topical compositions according to embodiments of the present invention can be any carrier known in the art for topically administering cosmetic or pharmaceutical compositions, including, but not limited to, cosmetically or pharmaceutically acceptable solvents, such as a polyalcohol or water; emulsions (either oil-in-water or water-in-oil emulsions), such as creams or lotions; micro emulsions; gels; ointments; liposomes; powders; and aqueous solutions or suspensions. The pharmaceutically or cosmetically acceptable carrier includes necessary and inert pharmaceutical excipients, including, but not limited to, binders, suspending agents, lubricants, flavorants, preservatives, dyes, and coatings.

Besides the carrier, the isolated BPs, such as the isolated HA and/or FA fractions, a topical composition according to an embodiment of the present invention also includes one or more other active ingredients that provide beneficial effects to the skin. For example, the composition can further include minerals, vitamins, enzymes, nutrients, other organic or inorganic chemicals for enhanced beneficial effects on the skin, e.g., for skin care, skin rejuvenation, skin nourishment, old age skin vitalization, etc. Due to the unique capability of the isolated BPs, e.g., as a transport medium, chelating agent, valance exchange media, and/or enzyme enhancer, the active ingredient can be administered to the skin at an amount lower than that used in the absence of the isolated BP to achieve the same efficacy. The active ingredient can also be administered to the skin at the same amount as that used in the absence of the isolated BP to achieve better efficacy.

According to embodiments of the present invention, the topical composition can include one or more active ingredients, including, but not limited to, one or more amino acids, vitamins, alpha-lipoic acids, alpha-hydroxy acids, hyaluronic acid, etc. for skin rejuvenation; one or more antioxidants, such as vitamins A, C, E, and extracts of pine bark, grape seed and green tea, catalase, superoxide dismutase and coenzyme Q-10 to protect the skin from free radicals and alleviate further cellular damage; one or more antimicrobial agents, such as terbinafine, kitoconazole, clotrimazole, naftifine, ciclopirox olamine, itraconazole, undecylenic acid to treat fungal infections such as onychomycosis; one or more of retinoids, tar, vitamin D3 and analogs thereof, corticosteroids, calcineurin inhibitors for treating psoriasis; one or more of metronidazole, azelaic acid, sodium sulfacetamide for treating rosacea; etc.

The pH of the topical composition of the invention are preferably within a physiologically acceptable pH, e.g., within the range of about 5 to about 8, such as 5, 5.5, 6, 6.5, 7, 7.5 or 8. To stabilize the pH, preferably, an effective amount of a buffer is included. In one embodiment, the buffering agent is present in the topical formulation in an amount of from about 0.05 to about 1 weight percent of the formulation. Acids or bases can be used to adjust the pH as needed.

The topical compositions can further include, e.g., one or more tonicity-adjusting agents, such as sodium chloride, potassium chloride, mannitol, dextrose, glycerin, and propylene glycol; one or more preservatives, such as parabens (e.g., methyl paraben and propyl paraben), benzalkonium chloride or chlorine dioxide; an antioxidant agent; a surfactant; an absorbent; one or more sun screen, such as zinc oxide; and a buffer system, such as ascorbic acid, sodium citrate and citric acid. The topical composition can also include other cosmetically or pharmaceutically acceptable excipients, such as those listed in Remington: The science and practice of pharmacy, 866-885 (Alfonso R. Gennaro ed. 19th ed. 1995); Ghosh, T. K.; et al. Transdermal and topical drug delivery systems (1997), hereby incorporated herein by reference.

In one embodiment of the present invention, a topical composition comprises, by weight, 0.5% to 30% of HA, 0.5% to 12% of FA, and 0.25% to 1% of UA, preferably 5-10% HA and 1-4% of FA, and trace amount of UA.

In one embodiment of the present invention, the isolated BP fraction used in the topical composition comprises an isolated FA fraction having a light molecule weight in the range of about 150 to 800 dalton, preferably 275 to about 500 daltons, more preferably 275 to 325 daltons of a nanocrystalline nature.

In another embodiment of the present invention, the isolated fulvic acid fraction used in the topical composition has an approximate formula of C₂₈H₁₆(COOH)₉(OH)₇(CO)₃.

In another embodiment of the present invention, the isolated fulvic acid fraction used in the topical composition has an approximate formula of C₃₅H₃₈N₁So₃O₃₆.

In another embodiment of the present invention, the isolated fulvic acid fraction used in the topical composition has 50.90% C, 44.75% O, 3.35% H, 0.75% N and 0.25% S.

In another embodiment of the present invention, the isolated fulvic acid fraction and/or humic acid fraction used in the topical composition has a cation exchange capacity potential of about 200 to 1000, preferably about 800, milliequevalents per 100 grams at a pH of 7.

In another embodiment of the present invention, the isolated isolated fulvic acid fraction used in the topical composition has a fulvic acid of formula (IV):

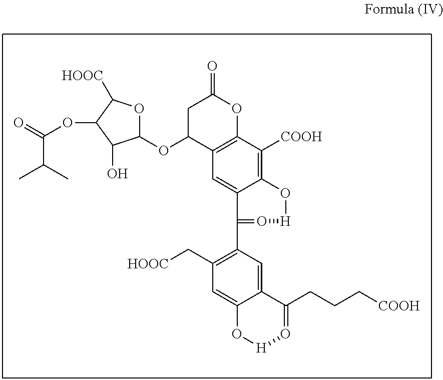

In another embodiment of the present invention, the isolated fulvic acid fraction used in the topical composition has a fulvic acid of formula (V):

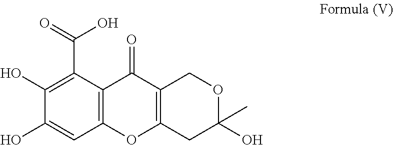 [SMILES: CC1(O)Cc2oc3cc(O)c(O)c(C(=O)O)c3c(=O)c2CO1]

Different isolated fulvic acid fractions can be used in the topical composition depending on factors such as the conditions to be treated, the properties of the other active or inactive ingredients in the formulation, etc.

In another embodiment of the present invention, the isolated humic acid fraction and/or the isolated BP fraction used in the topical composition is produced by a method comprising:

providing an aqueous slurry comprising a humified organic material (HOM);

applying an electromagnetic field to the aqueous slurry to obtain an electromagnetic field treated HOM; and

obtaining the isolated BP fraction from the electromagnetic field treated HOM.

In another embodiment of the present invention, the topical composition further comprises an effective amount of a water soluble zinc salt, preferably zinc sulfate. It was discovered that a topical composition according to an embodiment of the present invention without a water soluble zinc salt, such as zinc sulfate, has only minor effects on reducing wrinkle and lines, even though it has improved skin discoloration and general health of the subject. Without being bound by theory, the inventor posits that the water soluble zinc salt, such as a zinc sulfate, in the proposed formulation, is chelated into the polyelectrolyte acid and is metabolized into a bio-available anti-oxidant and cell stimulant.

According to embodiments of the present invention, the topical compositions improve skin health, reduces skin discoloration, enhances skin softness, relieves skin dryness, and reduces wrinkles, lines and age spots. In one embodiment, the topical composition comprises an isolated BP fraction, such as an isolated fulvic acid and/or humic acid fraction, ascorbic acid, tyrosine, zinc sulfate, and a preservative, at a pH of about 5 to 8, preferably 6.1 and 6.8. In another embodiment, the topical composition comprises an isolated BP fraction, such as an isolated FA and/or HA fraction, mineral oil from plant sources, vitamins A-C-D-E, cocoa butter, and oils of, Jojoba oil, sweet almond, coconut butter, grape seed, Emu oil plus Aloe Vera extract and Shea butter. In yet another embodiment, the topical composition comprises isolated BPs, such as isolated FA and HA fractions, mineral oil, Organic Beeswax, cocoa butter, Aloe Vera leaf extract, Shea Butter, vitamins A, B3, C, E, Jojoba Oil, olive leaf extract, sweet almond oil, avocado oil, avocado protein extract, and refined Emu oil.

According to other embodiments of the present invention, the topical compositions treat microbial infections, such as skin or nail fungal infections, e.g., onychomycosis, Athlete's foot, Jock itch, ringworm symptoms, etc. In one embodiment, the topical composition comprises an isolated BP fraction, such as an isolated FA and/or HA fraction, tea tree oil, butenafine hydrochloride, and one or more other plant oils or extracts. In another embodiment, the topical composition comprises an isolated BP fraction, such as an isolated FA and/or HA fraction, vitamins, undecylenic acid, and one or more other plant oils or extracts. In yet another embodiment, the topical composition comprises an isolated BP fraction, such as an isolated FA and/or HA fraction, clotrimazole, ciclopirox olamine, butenafine hydrochloride and lemon grass oil.

Topical compositions according to embodiments of the present invention can be used in products over a broad range of applications associated with topical skin and cellular abnormalities, including, but not limited to, face and neck moisturizers; hand and body lotions; severe skin damage repair aids; antifungal emulsions; face and body cleansers; natural essential oil lip balms; and numerous other specialty products such as; anti aging spot and skin discoloration creams, fine line erasers, etc.

The topical composition are prepared by mixing a pharmaceutically or cosmetically acceptable carrier with an effective amount of the isolated FA, HA or mixtures thereof according to known methods in the art, for example, methods provided by standard reference texts such as, Remington: The science and practice of pharmacy, 1577-1591, 1672-1673, 866-885 (Alfonso R. Gennaro ed. 19th ed. 1995); Ghosh, T. K.; et al. Transdermal and topical drug delivery systems (1997), both of which are hereby incorporated herein by reference.

In one embodiment of the invention, the topically administrable composition is contained within one suitable container, such as a dropper, a jar, or a tube with a suitable small orifice size, such as an extended tip tube, made of any pharmaceutically suitable material. The topical formulations of the invention can be filled and packaged into a plastic squeeze bottle or tube.

Preferably, instructions are packaged with the formulations of the invention, for example, a pamphlet or package label. The labeling instructions explain how to administer topical formulations of the invention, in an amount and for a period of time sufficient to improve the skin condition. Preferably, the label includes the dosage and administration instructions, the topical formulation's composition, etc.

In view of the present disclosure, the topical compositions of the invention can be topically applied directly to the affected area in any conventional manner known in the art, e.g., by dropper or applicator stick, as a mist via an aerosol applicator, via an intradermal or transdermal patch, or by simply spreading a formulation of the invention onto the affected area with fingers. Depending on the skin condition to be treated, typically, one to four applications per day are recommended during the term of treatment.

Methods of the present invention can be used in conjunction with one or more other treatments and compositions for improving a skin condition. The other treatment or composition can be administered to the subject simultaneously with, or in a sequence and within a time interval of, the administration of the composition of the present invention such that the active ingredients or agents can act together to improve the skin condition. The other treatment can be administered in the same or separate formulations at the same or different times.

Any suitable route of administration can be employed to deliver the additional treatment or medication including, but not limited to, oral, intraoral, rectal, parenteral, topical, epicutaneous, transdermal, subcutaneous, intramuscular, intranasal, sublingual, buccal, intradural, intraocular, intrarespiratory, or nasal inhalation.

This invention will be better understood by reference to the non-limiting examples that follow, but those skilled in the art will readily appreciate that the examples are only illustrative of the invention as described more fully in the claims which follow thereafter.

### Example 1

### Preparation of a Topical Composition

Preparation of a BP base: Base A

Desired amounts of isolated BPs, such as 0.5% to 30% of HA, 0.5% to 12% of FA, and 0.25% to 1% of UA, with the preferable percentages of 5-10% HA, 1-4% of FA, and trace amount of UA, are mixed into a solution of deionized water to produce “Base A.” All percentages of HA, FA and UA are by weight of Base A.

Preparation of a Mixture of BPs and Other Active Ingredients: Base B

To a solution of Base A, are added 2-20% of ascorbic acid, with a more preferable percentage of 5-15%; 1-10% of tyrosine, with 3-8 wt % being more preferable; and 0.5-5% of zinc sulfide, with 1-2% being more preferable, to produce “Base B.” All percentages of ascorbic acid, tyrosine and zinc sulfide are by weight of Base A used.

Preparation of a Topical Composition

Ingredients of Base B are blended until homogeneous and then added to a suitable tissue compatible vehicle, including, but not limited to, a hydrophilic lotion, cream, gel, mineral oil or any acceptable vehicle for topical application to skin. Suitable vehicles are available from commercial sources, such as Dermabase and Unibase formulations.

### Example 2

### A Double-Blinded Study on Skin Rejuvenation

A topical composition comprising ascorbic acid, tyrosine, zinc sulfate and a mixture of isolated HA and FA fractions was prepared. The topical composition was encapsulated in metered capsule form to ensure equal treatments in different participants.

Ten participants were chosen for a double-blinded study: 8 females and 2 males aging 50 to 72 years old. All participants were examined by an independent qualified dermatologist before (T0) and during the treatment (week 1 to week 6). A severity score from 1 to 10, with 10 being the worst condition, was assigned to the face or hand for wrinkles and skin lines. No grade was given for skin discoloration, age spots or other abnormalities. Each of the participants was given an identification letter, from A to J.

All participants received instructions on how the composition should be applied: for the face, applying the composition to the fingers of one hand, dabbing onto each cheek, forehead and around corners of the mouth, rubbing dabbed amount into skin in a circular motion, making 10 circles each; and for the back of hand: squeezing equal amounts on each hand back, followed by rubbing the backs of the two hands together in a circular motion one direction then the other. The participants were instructed to apply one capsule each to the face and hands twice daily: once in the morning and once before bed time, all after washing the respective areas. No effort to reapply during the day was attempted even when affected areas were washed.

The treatment was continued for 6 weeks in all participants, except one who stopped applying the composition to her skin after the third week due to a mild red rash appeared on cheek and at mouth edges as a reaction to tyrosine used in the formulation. The 9 participants were examined weekly for 6 or more weeks after the conclusion of the treatment.

Results of the Study are Summarized in Table 1

TABLE 1

| | T0 | 24 Hours | Week 1 | Week 2 | Week 4 | Week 6 |
|---|---|---|---|---|---|---|
| Patient | (Hand, | (Hand, | (Hand, | (Hand, | (Hand, | (Hand, |
| (age) | Face) | Face) | Face) | Face) | Face) | Face) |
| A (50) | 6, 6 | 5, 4 | 4, 4 | 3, 3 | 3, 2 | 2, 1 |
| B (58) | 6, 5 | 5, 5 | 4, 4 | 3, 3 | 3, 2 | 3, 2 |
| C (60) | 6, 5 | 4, 4 | 3, 4 | 2, 3 | 2, 3 | 2, 1 |
| D (65) | 6, 4 | 6, 4 | 5, 4 | 4, 3 | 2, 2 | 1, 1 |
| E (65) | 8, 6 | 7, 5 | 6, 5 | 5, 5 | 4, 4 | 3, 3 |
| F* (68) | 8, 8 | 6, 6 | 6, 4 | 5, 4 | 5, 3 | 4, 3 |
| G (69) | 8, 9 | 6, 7 | 6, 5 | 5, 4 | 4, 4 | 3, 3 |
| H (70) | 9, 7 | 7, 6 | 6, 5 | 5, 5 | 4, 4 | 3, 3 |
| I (65) | 8, 6 | 7, 6 | 6, 6 | 5, 5 | 4, 4 | 3, 3 |
| J (70) | 10, 9 | 8, 9 | 8, 8 | 6, 7 | 5, 6 | 5, 5 |

*Stopped applying the test composition to face and hands after the third week due to a mild red rash that appeared on cheek and at the mouth edges as a reaction to tyrosine used in the formulation.

All ten participants observed reduction in skin lines, wrinkles, discoloration, age spots, and thin skin texture. At the end of the 6 week treatment, 3 to 5 grades of improvement in wrinkles and skin lines were observed in all participants, including the one (F) who only received three week treatment, who had shown continued improvement of skin condition in the first few weeks after discontinuing the treatment. The suppleness of the epidural skin layer was dramatically improved in addition to the enhancement of the firmness of the dural under layer. The skin tone of each of the participants was healthy with good color and reduced brown discoloration.

Before the treatment (T0) Participant J's hands showed signs of severe damages from cracking deep enough to cause overburden scaring and heavy calluses. Heavy discoloration and large age spots were evident. At the end of week 6 treatment, the heavy discoloration was all gone, the age spots had diminished, and the leathery appearance of both hands and face were reduced by about 50% compared to that before the treatment.

No diminishing result in the improved skin conditions was observed up until week 3 after the treatment. Of the 9 participants, 6 showed little or no diminishing results, while 3 showed the beginning of the return of some wrinkles 6 weeks after the treatment, while skin color and suppleness remained the same as right after the treatment. Beginning in week 8 after the treatment, all participants showed signs of line and wrinkle return, but still no return of discoloration of age spots, leatheryness or loss of suppleness. All studies were concluded at week 14 from the beginning of the study.

Results from this study demonstrated that a topical composition according to an embodiment of the present invention can be used to improve skin conditions due to aging, such as to reduce wrinkles and lines, age spots, and increase the firmness of the skin.

### Example 3

### Antifungal Study

Four topical cream formulations shown below were made and tested in this study.

Formulation A, comprising:

Isolated HA fraction
Tea Tree oil
Butenafine hydrochloride
Lemon Oil
Pharmaceutically acceptable excipients

Formulation B, comprising:

Isolated FA fraction
Tea Tree oil
Vitamin E
Undecylenic acid
Jajoba Oil
Pharmaceutically acceptable excipients

Formulation C, comprising

FA fraction
Undecylenic acid
Urea
Almond Oil
Clove Oil
Tea Tree Oil
Pharmaceutically acceptable excipients

Formulation D, comprising

FA fraction
Clotrimazole
Ceclopirox Olamine
Tea Tree oil
Butenafine hydrochloride
Lemon grass Oil
Pharmaceutically acceptable excipients

Two patients each were selected for each of the four formulations, totaling 8 patients. All 8 patients were male and over 50 years of age. Each of the 8 patients had very similar conditions of fungal infection in multiple nails on both feet. The toe nails were much distorted, thick and with significant surrounding dead tissue.

All patient feet were scrubbed with an anti-microbial soap twice daily just prior to the application of the test formulation. The feet, especially the nail areas, were dried using disposable paper towels, followed by drying with a warm air dryer. Each of the test formulations was only applied topically to the left foot of each patient, leaving the right foot as a control. The two patients who received formulation B also received a liquid supplement twice a day in a dosage of 1 teaspoon in a glass of juice or cold water. The liquid supplement contained Fulvic Ultra, an ultra pure fulvic acid fraction prepared by the improved method suitable for oral administration, Yucca juice, vitamin C, E, and A.

All patients applied the treatment themselves after the first treatment, and kept a written log of appearance of the toe nails on both feet. Once every week, the patients were examined at a test site and both feet were photographed. The treatment was continued for 8 weeks.

Out of the 8 participants, 3 patents had complete remission of the infected area and showed a negative result from the scraping live fungus test in their feet received the treatment with the test formulations, while their control feet only showed slight (marginal) improvement, possibly due to the twice daily anti-fungal soap washing and the super drying effects. Three other patients had considerable improvement with an 80% cure rate in their feet received the treatment with the test formulations, but not in their control feet. The remaining two subjects also showed obvious improvements as compared to their untreated feet.

All 8 patients were supplied with sufficient amount of formulation A or B for one year and asked to return. Six patients reported complete cures with normal nails. One patient passed away and the other moved and could not be located.

This study demonstrated that compositions containing the isolated HA/FA fraction and one or more anti-fungal agent are highly effective in treating fungal infections.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A topical composition for improving a skin condition, comprising a cosmetically or pharmaceutically acceptable carrier, at least one active agent, and an effective amount of an isolated bioactive polyelectrolyte (BP) fraction comprising an isolated fulvic acid fraction, an isolated humic acid fraction, or an isolated mixture of fulvic acid and humic acid fraction, wherein the isolated fulvic acid fraction consists essentially of ionic nanocrystalline fulvic acid molecules, and the isolated humic acid fraction consists essentially of colloidal humic acid particles having an average particle size of about 1 to 10 microns in diameter.

2. The topical composition of claim 1, wherein the isolated fulvic acid fraction has an approximate formula of C₂₈H₁₆(COOH)₉(OH)₇(CO)₃.

3. The topical composition of claim 1, wherein the isolated fulvic acid fraction has an approximate formula of C₃₅H₃₈N₁So₃O₃₆.

4. The topical composition of claim 1, wherein the isolated fulvic acid fraction has a molecular weight range of about 150 to 800 dalton.

5. The topical composition of claim 1, wherein the isolated fulvic acid fraction has a molecular weight range of about 275 to 325 dalton.

6. The topical composition of claim 1, wherein the isolated fulvic acid fraction has 50.90% C, 44.75% O, 3.35% H, 0.75% N and 0.25% S.

7. The topical composition of claim 1, wherein the isolated fulvic acid fraction comprises an isolated fulvic acid of formula (IV):

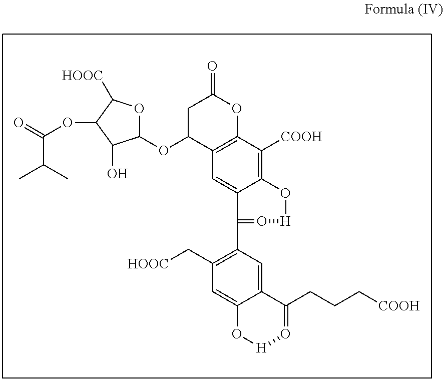

8. The topical composition of claim 1, wherein the isolated fulvic acid fraction comprises an isolated fulvic acid of formula (V):

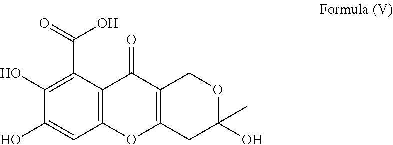 [SMILES: CC1(O)Cc2oc3cc(O)c(O)c(C(=O)O)c3c(=O)c2CO1]

9. The topical composition of claim 1, wherein the isolated BP fraction is produced by a method comprising:
providing an aqueous slurry comprising a humified organic material (HOM);
applying an electromagnetic field to the aqueous slurry to obtain an electromagnetic field treated HOM; and
isolating the BP fraction from the electromagnetic field treated HOM.

10. The topical composition of claim 1, further comprising an effective amount of zinc sulfate.

11. The topical composition of claim 1, comprising ascorbic acid, tyrosine, zinc sulfate, a preservative, and at least one of the isolated fulvic acid fraction and the isolated humic acid fraction.

12. The topical composition of claim 1, comprising the therapeutically acceptable carrier, an anti-fungal agent and at least one of the isolated fulvic acid fraction and the isolated humic acid fraction.

13. A method for improving a skin condition in a subject, comprising topically applying to a skin area of the subject in need of the improvement a topical composition of claim 1.

14. The method of claim 13, wherein the isolated fulvic acid fraction has an approximate formula of C₂₈H₁₆(COOH)₉(OH)₇(CO)₃.

15. The method of claim 13, wherein the isolated fulvic acid fraction has an approximate formula of C₃₅H₃₈N₁So₃O₃₆.

16. The method of claim 13, wherein the isolated fulvic acid fraction has a molecular weight range of about 150 to 800 dalton.

17. The method of claim 13, wherein the isolated fulvic acid fraction has a molecular weight range of about 275 to 500 dalton.

18. The method of claim 13, wherein the isolated fulvic acid fraction has 50.90% C, 44.75% O, 3.35% H, 0.75% N and 0.25% S.

19. The method of claim 13, wherein the isolated fulvic acid fraction comprises a fulvic acid of formula (IV):

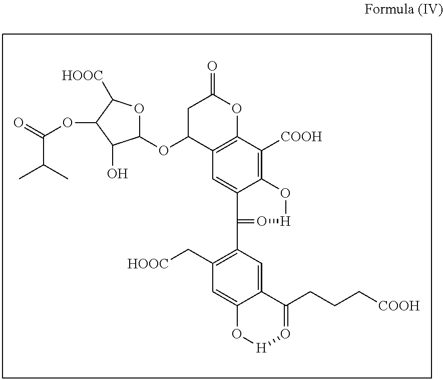

20. The method of claim 13, wherein the isolated fulvic acid fraction comprises a fulvic acid of formula (V):

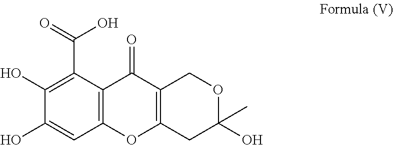 [SMILES: CC1(O)Cc2oc3cc(O)c(O)c(C(=O)O)c3c(=O)c2CO1]

21. The method of claim 13, wherein the isolated BP fraction is produced by a method comprising:
providing an aqueous slurry comprising a humified organic material (HOM);
applying an electromagnetic field to the aqueous slurry to obtain an electromagnetic field treated HOM; and
isolating the BP fraction from the electromagnetic field treated HOM.

22. The method of claim 13, wherein the composition further comprises an effective amount of zinc sulfate.

23. The method of claim 22, wherein the composition comprises ascorbic acid, tyrosine, zinc sulfate, a preservative, and at least one of the isolated fulvic acid fraction and the isolated humic acid fraction.

24. The method of claim 13, wherein the skin condition is a fungal infection, and the topical composition comprises the therapeutically acceptable carrier, an anti-fungal agent and at least one of the isolated fulvic acid fraction and the isolated humic acid fraction.
